(19) United States
(12) Patent Application Publication
(10) Pub. No.: US 2023/0248219 A1
(43) Pub. Date: Aug. 10, 2023

(54) VALVE ASSEMBLY

(71) Applicant: CAMBRIDGE UNIVERSITY HOSPITAL NHS FOUNDATION TRUST, Cambridgeshire (GB)

(72) Inventors: Andrew Hindmarsh, Cambridgeshire (GB); Gareth Corbett, Cambridgeshire (GB)

(21) Appl. No.: 18/014,913

(22) PCT Filed: Jul. 6, 2021
(86) PCT No.: PCT/GB2021/051710

§ 371 Date: Jan. 6, 2023

(30) Foreign Application Priority Data
Jul. 8, 2020 (GB) 2010491.5

(51) Int. Cl. A61B 1/00; A61B 46/13; A61B 46/00; A61M 16/06; A61B 17/34

(52) U.S. Cl. CPC A61B 1/00142; A61B 46/13; A61B 46/30; A61B 1/00128; A61M 16/0622; A61B 17/3498

(57) ABSTRACT

A valve assembly (1, 12, 21, 31, 44, 52, 60) includes a hollow tube (2). The valve assembly (1, 12, 21, 31, 44, 52, 60) also includes a first set of one or more valves (3) 5 arranged within the hollow tube (2). The first set of valves (3) is configured to permit sliding passage of a substantially cylindrical object (4). The valve assembly (1, 12, 21, 31, 44, 52, 60) also includes a second set of valves (5) arranged within the hollow tube (2) and spaced apart from the first set of valves to (3) define a chamber. The second set of valves (5) are configured to permit sliding passage of the cylindrical object (4). The 10 valve assembly (1, 12, 21, 31, 44, 52, 60) also includes one or more ultraviolet light sources (10) within the chamber (6) and/or a first port (13) connecting the chamber (6) to the exterior of the hollow tube (2) and configured for connection to a fluid flow path (14) for supply and/or extraction of fluid (19).

### FIELD OF THE INVENTION

The present invention relates to a valve assembly for medical applications. In particular the present invention relates to valve assemblies for reducing or preventing aerosolisation of infectious agents such as viruses and/or bacteria during endoscopy and/or keyhole surgery.

### BACKGROUND

The emergence of severe acute respiratory syndrome coronavirus 2 (SARS-CoV-2) and the resulting infectious disease COVID-19 has highlighted significant infection control challenges for healthcare providers trying to provide procedures and services, whether related to COVID-19 or not. In particular, challenges in preventing cross-infection between patients and staff have risen to prominence.

Procedures that may cause aerosolisation of secretions from the throat or from the bowel may be considered to be especially high risk for transmission and include, for example, Endoscopy (gastroscopy, colonoscopy, bronchoscopy, nasopharyngoscopy), Bowel surgery (especially laparoscopy), Tracheal intubation and so forth.

It is currently considered that active COVID-19 may be present in the gastro-intestinal (GI) tract and faeces for many weeks in patients who were infected with COVID-19 but subsequently recovered. This may have the consequence that screening of patients for symptoms, or even using diagnostic tests, may be insufficient to prevent aerosolisation of infectious particles

Although the COVID-19 pandemic has exposed aerosol generating procedures (AGP) as a significant infection control issue, the infection control implications of aerosol generating procedures are not limited to COVID-19 and would be expected to be relevant to any viral or bacterial agent capable of transmission via aerosolisation.

Health care providers have tried to use containment devices to contain aerosolised droplets, for example Michele Marchese, Annalisa Capannolo, Loreto Lombardi, Michela Di Carlo, Franco Marinangeli, and Pierfrancesco Fusco, “Use of a modified ventilation mask to avoid aerosolizing spread of droplets for short endoscopic procedures during coronavirus COVID-19 outbreak”, Gastrointestinal Endoscopy, letters to the editor, 2 Apr. 2020 (DOI:https://doi.org/10.1016/j.gie.2020.03.3853), describe an anaesthetic face mask modified to allow passage of an endoscope.

Devices for improving oxygen flow during endoscopy have been described, for example, U.S. Pat. No. 5,431,158 A describes an endoscopic breathing mask configured to fit over the mouth and nose of a patient, which has an air tube positioned so that oxygen can be continuously and controllably introduced into the mask.

Garments for use in connection with endoscopy have been described, for example, EP 1657988 B1 describes a garment to be worn by a patient during endoscopy, which is cut and sized to fit snugly around a pelvic area of the patient, such that when the garment is worn by the patient, the fabric defines two leg holes and an aperture, which is positioned adjacent to a body orifice of the patient so as to permit insertion of an endoscope through the aperture into the body orifice.

### SUMMARY

According to a first aspect of the invention there is provided a valve assembly including a hollow tube. The valve assembly also includes a first set of one or more valves arranged within the hollow tube. The first set of valves is configured to permit sliding passage of a substantially cylindrical object. The valve assembly also includes a second set of valves arranged within the hollow tube and spaced apart from the first set of valves to define a chamber. The second set of valves are configured to permit sliding passage of a cylindrical object. The valve assembly also includes one or more ultraviolet light sources within the chamber and/or a first port connecting the chamber to the exterior of the hollow tube and configured for connection to a fluid flow path for supply and/or extraction of fluid.

The hollow tube may be substantially cylindrical. Substantially cylindrical may include cylindrical. The hollow tube may have a cross-section which is elliptical, square, rectangular, pentagonal, hexagonal, or any other regular or irregular shape.

With the exception of the first port when included, the chamber may be substantially airtight when a cylindrical object is received through the hollow tube. The hollow tube may include, or be formed from, rigid plastic.

The first set of valves may form a substantially airtight (alternatively fluid-tight) seal around the substantially cylindrical object. The second set of valves may form a substantially airtight (alternatively fluid-tight) seal around the substantially cylindrical object.

An ultraviolet light source may emit light including, or centred about, the wavelength range between and including 200 nm and 280 nm. An ultraviolet light source may include, or take the form of, one or more light-emitting diodes.

A fluid may include a gas. A fluid may include a liquid. The fluid flow path may take the form of a tube or pipe.

The first port may be a first valved port. The first valved port may include, or take the form of, a duck billed valve configured to close the first port when the first port is disconnected and/or when the first port does not receive a tube or pipe. The first valved port may include, or take the form of, one or more lumens configured to close the first port when the first port is disconnected. The first valved port may include, or take the form of, a first one-way valved port configured to permit fluids to exit the chamber via the first port.

The valve assembly may include the first port, and may further include a second port connecting the chamber to the exterior of the hollow tube and configured for connection to a fluid flow path for supply and/or extraction of fluid.

A liquid may include a viricidal substance or composition. The second port may be a second valved port. The second valved port may include, or take the form of, a duck billed valve configured to close the second port when the second port is disconnected and/or when the second port does not receive a tube or pipe. The second valved port may include, or take the form of, one or more lumens configured to close the second port when the second port is disconnected. The second valved port may include, or take the form of, a second one-way valved port configured to permit fluids to enter the chamber via the second port.

The first set of valves may include two or more valves. The first set of valves may include three valves. The first set of valves may include four valves. The first set of valves may include five or more valves.

The second set of valves may include two or more valves. The second set of valves may include three valves. The second set of valves may include four valves. The second set of valves may include five or more valves.

The first set of valves may include at least one septum valve. The second set of valves may include at least one septum valve. Each septum valve may include three, four, five or six leaves. Each septum valve may include more than six leaves. Each septum valve may form a seal around the substantially cylindrical object when the substantially cylindrical object slides through the valve assembly.

The first set of valves may include at least one duck-billed valve. The second set of valves may include at least one duck-billed valve. Each duck billed valve may be open when the substantially cylindrical object slides in a first direction and/or a second direction opposite to the first direction. Each duck billed valve may form a seal when the substantially cylindrical object is removed from the valve assembly.

The first set of valves may include at least one O-ring. The second set of valves may include at one least O-ring.

According to a second aspect of the invention there is provided a valve assembly including a hollow tube defining a channel between a first opening and a second opening. The valve assembly also includes a third set of one or more valves arranged within the hollow tube. The third set of valves is configured to permit sliding passage of a substantially cylindrical object. The valve assembly also includes a first port connecting the channel to the exterior of the hollow tube. The first port is configured for connection to a fluid flow path for extraction of gas. The first port is disposed between the third set of valves and the first opening.

The third set of one of more valves may be proximate to the second opening of the hollow tube. The third set of one of more valves may be closer to the second opening of the hollow tube than to the first opening of the hollow tube. The hollow tube may be substantially cylindrical. Substantially cylindrical may include cylindrical. The hollow tube may have a cross-section which is elliptical, square, rectangular, pentagonal, hexagonal, or any other regular or irregular shape.

The hollow tube may include, or be formed from, rigid plastic. The third set of valves may form a substantially airtight (alternatively fluid-tight) seal around the substantially cylindrical object.

The first port may be a first valved port. The first valved port may include, or take the form of, a duck billed valve configured to close the first port when the first port is disconnected and/or when the first port does not receive a tube or pipe. The first valved port may include, or take the form of, one or more lumens configured to close the first port when the second port is disconnected. The first valved port may include, or take the form of, a first one-way valved port configured to permit fluids to exit the channel via the first port.

The valve assembly may also include a second port connecting the channel to the exterior of the hollow tube. The second port may be configured for connection to a fluid flow path for supply of fluid. The second port may be disposed between the first end and the first port. The second port may be proximate to the first end of the hollow tube. The second port may be closer to the first end of the hollow tube than to the first port.

The second port may be a second valved port. The second valved port may include, or take the form of, a duck billed valve configured to close the second port when the second port is disconnected and/or when the second port does not receive a tube or pipe. The second valved port may include, or take the form of, one or more lumens configured to close the second port when the second port is disconnected. The second valved port may include, or take the form of, a second one-way valved port configured to permit fluids to enter the channel via the second port.

The third set of valves may include two or more valves. The third set of valves may include three valves. The third set of valves may include four valves. The third set of valves may include five or more valves.

The third set of valves may include at least one septum valve. Each septum valve may include three, four, five or six leaves. Each septum valve may include more than six leaves. Each septum valve may form a seal around the substantially cylindrical object when the substantially cylindrical object slides through the valve assembly.

The third set of valves may include at least one duck-billed valve. Each duck billed valve may be open when the substantially cylindrical object slides in a first direction and/or a second direction opposite to the first direction. Each duck billed valve may form a seal when the substantially cylindrical object is removed from the valve assembly.

The third set of valves may include at least one O-ring.

Hereinafter, references to the valve assembly refer, except where features are evidently incompatible, to the valve assembly according to the first aspect and/or the valve assembly according to the second aspect.

A mask for covering the mouth and nose of a patient may include the valve assembly. The mask may be formed from a gas impermeable material and may be configured to form an airtight seal around a periphery of the mask. The hollow tube of the valve may pass through the mask. The mask may also include a third port connecting an inner surface of the mask to an outer surface of the mask and including a first filter. The first filter may be configured to remove aerosolised particles.

The third port may take the form of a hole in the mask material. The first filter may be joined to the mask. The first filter may be integrated with the mask. The inner surface of the mask may be a surface configured to face a patient in use and the outer surface may be a surface configured to face away from a patient in use. The valve assembly may be integrated with, or connected to, the mask so that a join between the mask and the valve assembly is air-tight.

Removing particles may mean entrapping particles. The first filter may be a high-efficiency particulate air (HEPA) filter. The first filter may be an ultra-low particulate air (ULPA) filter. The first filter may be configured to remove particles having a dimension greater than or equal to 500 nm, greater than or equal to 300 nm, greater than or equal to 200 nm, greater than or equal to 150 nm, or greater than or equal to 125 nm. The first filter may be configured to remove virus molecules. The first filter may be configured to remove COVID-19. A portion of the mask including the valve assembly may include, or be formed from, a flexible material to enable positioning and/or adjustment of the position of the valve assembly relative to the mouth or nose of a patient. A portion of the mask including the valve assembly may include, or be formed from, a transparent flexible material.

The third port may include, or take the form of, a third one-way valved port configured to permit gas to pass in a direction from the inner surface to the outer surface.

The mask may also include a fourth port connecting the inner surface of the mask to the outer surface of the mask for delivery of oxygen to a patient wearing the mask. The fourth port may be a valved port. The fourth valved port may include a duck billed valve configured to close the fourth port when fourth first port is disconnected and/or when the fourth port does not receive a tube or pipe. The fourth valved port may include one or more lumens configured to close the fourth port when the fourth port is disconnected. The fourth port may include, or take the form of, a one-way valved port configured to permit gas to pass in a direction from the outer surface to the inner surface.

The mask may also include a suction catheter integrated with the mask and configured for suction of fluids through the mask. The suction catheter may be integrated with, or connected to, the mask so that a join between the mask and the suction catheter is air-tight.

A portion of the mask including the suction catheter may include, or be formed from, a flexible material to enable positioning and/or adjustment of the position of the suction catheter relative to the mouth or nose of a patient. A portion of the mask including the suction catheter may include, or be formed from, a transparent flexible material.

The hollow tube of the valve assembly included in the mask may also include a mouth guard. The hollow tube of the valve assembly may be connected to a mouth guard.

A garment may include the valve assembly. The garment may be configured for forming a first airtight seal around the waist of a patient and second and third airtight seals around corresponding legs of a patient so as to enclose a volume between the patient, the garment and the first to third airtight seals. The garment may be formed from a gas impermeable material. The hollow tube of the valve may passes through the garment. The garment may also include a fifth port connecting an inner surface of the garment to an outer surface of the garment and including a second filter. The second filter may be configured to remove aerosolised particles.

The fifth port may take the form of a hole in the garment material. The second filter may be joined to the garment. The second filter may be integrated with the mask. The inner surface of the garment may be a surface configured to face a patient in use and the outer surface may be a surface configured to face away from a patient in use. The valve assembly is integrated with, or connected to, the garment so that a join between the garment and the valve assembly is air-tight.

Removing particles may mean entrapping particles. The second filter may be a high-efficiency particulate air (HEPA) filter. The second filter may be an ultra-low particulate air (ULPA) filter. The second filter may be configured to entrap particles having a dimension greater than or equal to 500 nm, greater than or equal to 300 nm, greater than or equal to 200 nm, greater than or equal to 150 nm, or greater than or equal to 125 nm. The second filter may be configured to remove virus molecules. The second filter may be configured to remove COVID-19. A portion of the garment including the valve assembly may include, or be formed from, a flexible material to enable positioning and/or adjustment of the position of the valve assembly relative to the anus of a patient. A portion of the garment including the valve assembly may include, or be formed from, a transparent flexible material.

The garment may also include a sealed, flexible projection arranged to be positioned proximate to the patient's anus when the garment is worn. The projection may be configured to receive a finger in order to perform a digital rectal examination of the patient. A portion of the garment including the flexible projection may include, or be formed from, a flexible material to enable positioning and/or adjustment of the position of the flexible projection relative to the anus of a patient. A portion of the garment including the flexible projection may include, or be formed from, a transparent flexible material.

An endoscope may include the valve assembly, and a working channel. The hollow tube may form an airtight seal to a user end of the working channel. The valve assembly may be connected to, or integrated with, a port providing access to the working channel. The valve assembly may replace a currently available endoscopic biopsy channel seal. The valve assembly may be configured for introducing endoscopic consumable instruments such as biopsy forceps into the working channel. Any ports or seals of the endoscope may include, or be accessed via, a valve assembly.

A trocar may include the valve assembly. A cannula of the trocar may be configured to be accessed via the valve assembly.

An apparatus may include the valve assembly, the mask, the garment, the endoscope or the trocar. The apparatus may also include a source of suction connected to the first port of the valve assembly. The source of suction may include, or take the form of, a pump. The apparatus may also include a fluid source connected to the second port of the valve assembly.

The fluid source may supply a gas. The fluid source may supply a liquid. A liquid may include a viricidal substance or composition. The fluid source may be pressurised. The fluid source may be a pump or a compressor.

The apparatus may also include a third filter configured to filter fluid between the first port and the source of suction to remove aerosolised particles. Removing particles may mean entrapping particles. The third filter may be a high-efficiency particulate air (HEPA) filter. The third filter may be an ultra-low particulate air (ULPA) filter. The third filter may be configured to remove particles having a dimension greater than or equal to 500 nm, greater than or equal to 300 nm, greater than or equal to 200 nm, greater than or equal to 150 nm, or greater than or equal to 125 nm. The third filter may be configured to remove virus molecules. The third filter may be configured to remove COVID-19.

The source of suction may take the form of an inlet to a closed loop and the fluid source may take the form of an outlet of the closed loop. The closed loop may include a pump and a fourth filter configured to remove aerosolised particles.

Removing particles may mean entrapping particles. The fourth filter may be a high-efficiency particulate air (HEPA) filter. The fourth filter may be an ultra-low particulate air (ULPA) filter. The fourth filter may be configured to remove particles having a dimension greater than or equal to 500 nm, greater than or equal to 300 nm, greater than or equal to 200 nm, greater than or equal to 150 nm, or greater than or equal to 125 nm. The fourth filter may be configured to remove virus molecules. The fourth filter may be configured to remove COVID-19. The closed loop and the chamber of the valve assembly may be filled with a liquid including a viricidal substance or composition, for example a solution of soap in water.

According to a third aspect of the invention, there is provided a method including connecting a first port of the valve assembly, the mask, the garment, the endoscope, or the trocar to a source of suction. The source of suction may include, or take the form of, a pump.

The method may also include connecting a fluid source to the second port of the valve assembly. The fluid source may supply a gas. The fluid source may supply a liquid. A liquid may include a viricidal substance or composition. The fluid source may be pressurised. The fluid source may be a pump or a compressor.

The method may also include connecting a third filter between the first port and the source of suction to remove aerosolised particles. Removing particles may mean entrapping particles. The third filter may be a high-efficiency particulate air (HEPA) filter. The third filter may be an ultra-low particulate air (ULPA) filter. The third filter may be configured to remove particles having a dimension greater than or equal to 500 nm, greater than or equal to 300 nm, greater than or equal to 200 nm, greater than or equal to 150 nm, or greater than or equal to 125 nm. The third filter may be configured to remove virus molecules. The third filter may be configured to remove COVID-19.

The source of suction may take the form of an inlet to a closed loop and the fluid source may take the form of an outlet of the closed loop. The closed loop may include a pump and a fourth filter configured to remove aerosolised particles. Removing particles may mean entrapping particles. The fourth filter may be a high-efficiency particulate air (HEPA) filter. The fourth filter may be an ultra-low particulate air (ULPA) filter. The fourth filter may be configured to remove particles having a dimension greater than or equal to 500 nm, greater than or equal to 300 nm, greater than or equal to 200 nm, greater than or equal to 150 nm, or greater than or equal to 125 nm. The fourth filter may be configured to remove virus molecules. The fourth filter may be configured to remove COVID-19. The closed loop and the chamber of the valve assembly may be filled with a liquid including a viricidal substance or composition, for example a solution of soap in water.

According to a fourth aspect of the invention, there is provided use of the valve assembly, the mask, the garment, the endoscope and/or the trocar in a medical procedure.

The valve assembly, the mask and/or the endoscope may be used in an endoscopy procedure. The valve assembly, the garment and/or the endoscope may be used in a colonoscopy procedure. The valve assembly, the endoscope and/or the trocar may be used in a keyhole surgery procedure.

The valve assembly may be used in any lower gastro-intestinal endoscopy procedures such as, for example, prostoscopy, rigid and flexible sigmoidosopy, colonoscopy and so forth. The valve assembly may be used in any upper gastrointestinal endoscopy procedures such as, for example, gastroscopy, nasendoscopy, enteroscopy, endoscopic ultrasound (EUS), endoscopic retrograde cholangio-pancreatography (ERCP) and so forth. The valve assembly may be used in any endoscopic respiratory procedures such as, for example, bronchoscopy, endobronchial ultrasound (EBUS), pharyngoscopy and so forth.

The valve assembly may be used in keyhole surgery such as, for example, laparoscopy, thoracoscopy, or similar minimally invasive procedures which utilise trocars/ports for access to the body.

The valve assembly may be used for transrectal ultrasound (TRUS) guided Prostate biopsy (TRUS) procedures.

The valve assembly may be used for extubation and other manoeuvres or procedures with endotracheal tubes such as, for example, changing ventilators and so forth.

### BRIEF DESCRIPTION OF THE DRAWINGS

Certain embodiments of the present invention will now be described, by way of example, with reference to the accompanying drawings in which:

FIG. 1 is a schematic cross-section of a first valve assembly;

FIG. 2 is a schematic cross-section of a second valve assembly;

FIG. 3 is a schematic cross-section of a third valve assembly in a first configuration;

FIG. 4 is a schematic cross-section of the third valve assembly in a second configuration;

FIG. 5A is a schematic cross-section of a fourth valve assembly along a central axis, FIG. 5B is a view along the central axis of the fourth valve assembly;

FIGS. 6A, 6B and 6C schematically illustrate septum valves having varying numbers of leaves;

FIGS. 7A to 8B schematically illustrate operation of a septum valve;

FIGS. 9A to 10B schematically illustration operation of a duck billed valve;

FIG. 11A is a schematic cross-section of an O-ring valve along a central axis, FIG. 11B is a view along the central axis of an O-ring valve;

FIG. 12A is a schematic cross-section of a fifth valve assembly, FIG. 12B is a schematic cross-section of the fifth valve assembly along the line labelled B-B′ in FIG. 12A;

FIG. 13A is a schematic cross-section of a sixth valve assembly, FIG. 13B is a schematic cross-section of the sixth valve assembly along the line labelled C-C′ in FIG. 13A;

FIG. 14A is a schematic front view of a mask including a valve assembly, FIGS. 14B and 14C are schematic side views of the mask;

FIG. 15A is a schematic view of a garment including a valve assembly, FIG. 15B is a schematic cross-section along the line labelled D-D′ in FIG. 15A;

FIG. 16 is a schematic cross-section of an endoscope including a valve assembly; and

FIG. 17 is a schematic cross-section of a trocar including a valve assembly.

### DETAILED DESCRIPTION OF CERTAIN EMBODIMENTS

In the following, like parts are denoted by like reference numbers.

Aerosolisation of viral droplets in the range from tens to hundreds of nm poses a risk of infection to those in close proximity to the site of aerosolisation. Many medical procedures have the potential to generate aerosol droplets including procedures such as, for example, endotracheal intubation, upper gastro-intestinal (GI) endoscopy and bronchoscopy amongst others. In addition there are concerns that lower GI endoscopic procedures and laparoscopy may also lead to generation of aerosolised virus.

Potentially aerosol generating procedures (AGP) include a large number and range of commonly performed emergency and elective procedures, and when an infectious agent such as COVID-19 is circulating in a population the need for effective infection control means that any patient must be considered as potentially being infected. The extent and risk of AGP is not yet fully known, for example, the infectivity (risk of infection) from an AGP is not known, but values have been reported to be anywhere between 1% and 50% likelihood. If this is combined with a situation in which testing of the population is imperfect, for example there is a significant false negative rate for swabbing specifically for COVID-19, means that there cannot be certainty that a patient is not infectious and thus AGP will remain a significant infection control risk until a solution that safely contains aerosolised droplets/particles generated during procedures can be implemented.

Modelling suggests that there could be recurrent waves of COVID-19 infection until 2028. Even once COVID-19 has been brought under control, there will remain the possibility of further outbreaks of infectious agents which could spread via AGP. Health care providers need solutions which may help to mitigate the risks of AGP transmitting infectious agents, for example COVID-19, between patients and staff so they can begin to reinstate ‘normal’ health services. This is necessary because regular healthcare and screenings cannot simply stop during an epidemic or pandemic of an infectious disease.

GI endoscopies (gastroscopy and colonoscopy) are common procedures, for example, approximately 1.6 million and approximately 38M are performed in the UK and US respectively every year. The division between gastroscopy and colonoscopy procedures is approximately 50:50.

Prior to COVID-19 a routine endoscopy session at a UK tertiary care hospital might deliver 12 gastroscopies, or 6 colonoscopies, or a combination of these. In the context of COVID-19 these procedures are considered high risk for aerosol transmission and infection between patients and staff. Staff can wear protective equipment during the procedure, however, sufficient time must be left between procedures to allow air changes in the endoscopy room to clear any virus. If an endoscopy suite has a standard ventilation system, then the time taken for the air to clear may be approximately one and a half hours. Consequently only 2 endoscopies can be performed per session (instead of the 12 gastroscopies or 6 colonoscopies performed pre COVID-19). Such considerations have significant implications for patient capacity and the practicality of delivering anything approaching a ‘normal’ service.

Gastroscopy is a diagnostic and/or therapeutic procedure performed using an endoscope. An endoscope is a long flexible plastic tube which utilises fibreoptic technology to transmit an image from its tip. The endoscope is inserted through a patient's mouth, and allows direct visual examination of gullet, stomach and duodenum. During a gastroscopy, the patient is provided supplemental oxygen via a nasal cannula, and the patient's mouth is accessible for suction of secretions. Gastroscopy procedures are considered a high risk for aerosolisation.

Colonoscopy is a diagnostic and/or therapeutic procedure performed using an endoscope. Colonoscopy involves that direct examination of rectum, colon and terminal ileum. The patient may be awake or sedated. Colonoscopy procedures are considered a high risk for aerosolisation. It is believed that COVID-19 may remain active in the bowel for many weeks after a patient has recovered from infection.

In an attempt to mitigate some of the risks, issues and problems associated with potentially aerosol generating endoscopic procedures, the inventors have developed a valve assembly which may be used to enable use of an endoscope for and/or in a variety of procedures, whilst substantially containing aerosolised droplets/particles. The valve assembly described herein may be incorporated into masks, garments, endoscopes, trocars, and/or other equipment for use in endoscopic procedures. The valve assembly described herein may also find use in procedures such transrectal ultrasound (TRUS) guided Prostate biopsy (TRUS) procedures, extubation and/or other manoeuvres or procedures with endotracheal tubes such as, for example, changing ventilators and so forth.

Herein the terms gas impermeable, gas-tight, airtight and so forth refer to materials or objects which substantially do not permit gases to pass. Such materials may still permit diffusion of gas molecules, and would still be considered gas impermeable for the present purposes.

### First Valve Assembly

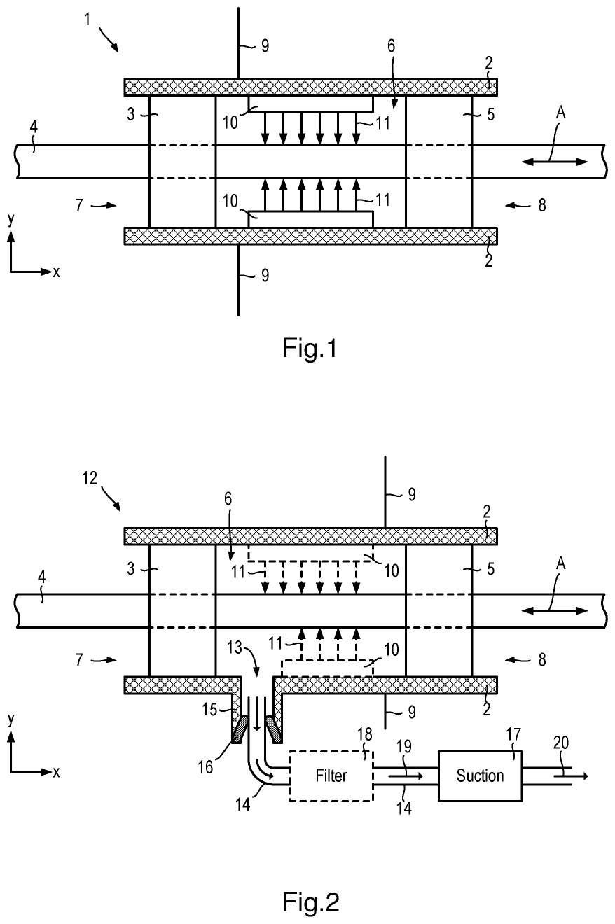

Referring to FIG. 1, a schematic cross-section of a first valve assembly 1 is shown.

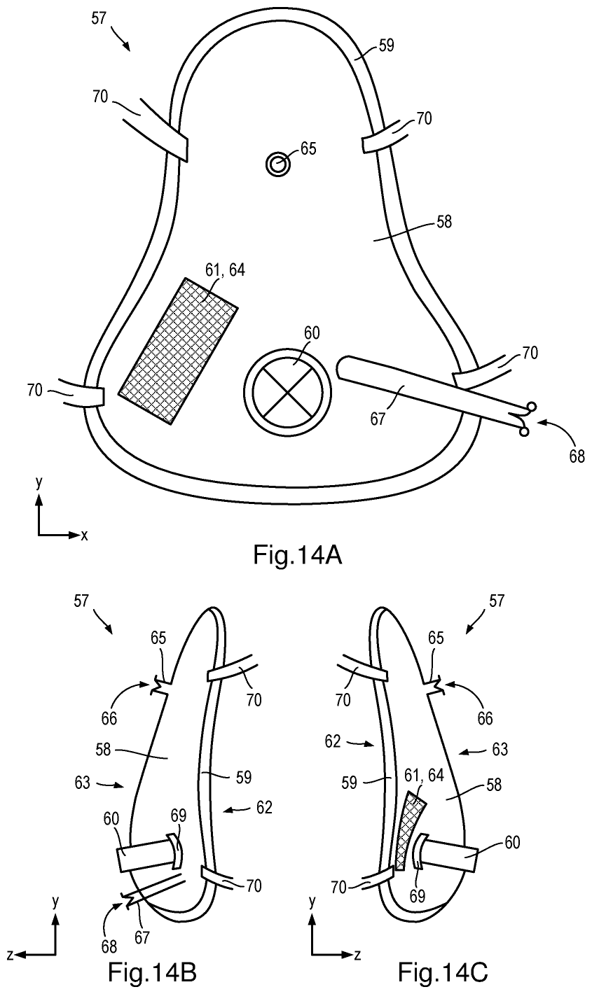

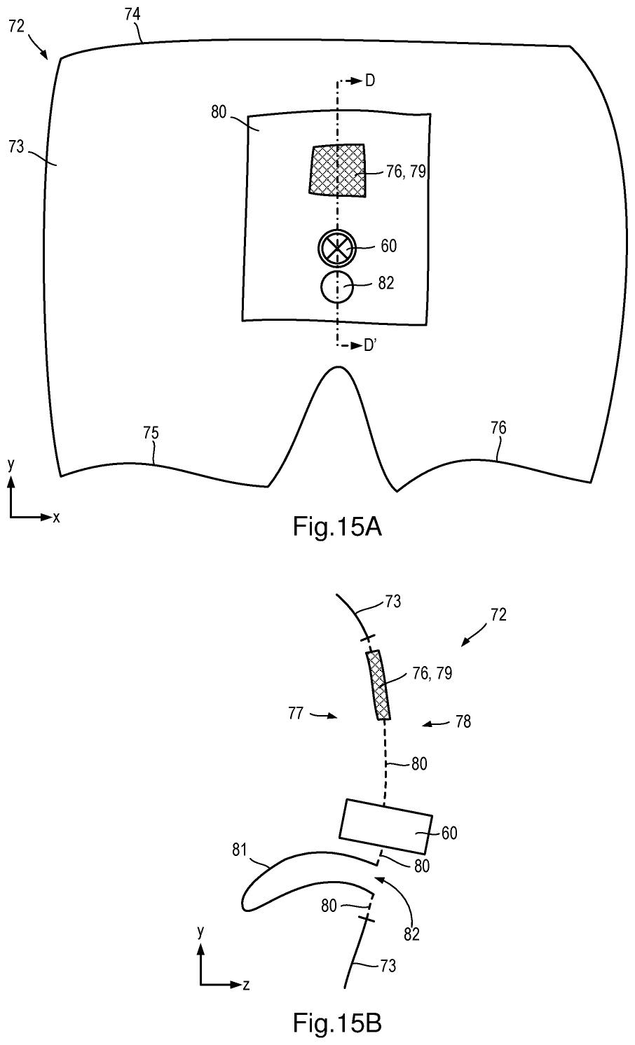

The first valve assembly 1 includes a hollow tube 2. A first set of one or more valves 3 is arranged within the hollow tube 2. The first set of valves 3 is configured to permit sliding passage of a substantially cylindrical object 4 along a first direction, x, as illustrated by the arrow labelled “A”. A second set of valves 5 is arranged within the hollow tube 4, and is spaced apart along the first direction x from the first set of valves 3 to define a chamber 6. The second set of valves 5 is also configured to permit sliding passage of the cylindrical object 4 along the first direction. The hollow tube 2 has a first opening 7 providing access to the first set of valves 3 and a second opening 8 providing access to the second set of valves. In use, the hollow tube 2 may pass through an air/fluid-tight barrier 9 (for example a mask in FIG. 14, a garment in FIG. 15, and so forth), so that the first opening 7 is on a clinician side of the barrier 9 and the second opening 8 is on a patient side of the barrier 9. In use, a cylindrical object 4, for example and endoscope or similar medical device, may be inserted through the first opening 7, to pass through the first set of valves 3, the chamber 6 and the second set of valves 5 to emerge through the second opening 8 onto the patient side of the barrier 9.

The second set of valves 5 forms a seal around the cylindrical object 4, and reduces fluids and/or droplets from the patient side of the barrier 9 from entering the chamber 6. Any fluids and/or droplets which do enter the chamber 6 are sterilised by an anti-viral mechanism in the form of one or more ultraviolet light sources 10 disposed within the chamber and arranged to irradiate the chamber 6 and any cylindrical object 4 passing through the chamber 6 using ultraviolet (UV) light 11. Each ultraviolet light source 10 preferably emits light 11 including, or centred about, the wavelength range between and including 200 nm and 280 nm. In other words, UV-C wavelengths. Ultraviolet light 11 of sufficient intensity may be effective in killing/denaturing/destroying viruses and/or bacteria. Each ultraviolet light source may include, or take the form of, a light-emitting diode. However, other methods of introducing UV light 11 into the chamber 6 may be used, for example, one or more fibre-optic bundles may be used to deliver UV light 11 from an external UV light source (not shown) into the chamber 6.

The first set of valves 3 also form a seal around the cylindrical object 4, so that by the time a portion of the cylindrical object 4 has been withdrawn from the first opening 7, it will have passed through two seals and a biocidal “air-lock” in the form of the chamber 6 including the UV-light sources 10. In this way, escape of fluids and/or particles containing infectious agents may be reduced or eliminated, since any fluids and/or particles which do manage to pass both sets of valves 3, 5 will also have been irradiated using UV light 11. The combination of the barrier 9 between the patient and staff with the valve assembly 1 may enable endoscopic procedures (or other procedures performed using substantially cylindrical objects 4) to be carried out whilst containing potentially infectious fluids and/or aerosolised particles/droplets on a patient side of the barrier 9. In addition to reducing the risk of direct infection of medical staff, this may also enable a higher throughput by reducing the need for extensive ventilation to change the air in a room to avoid cross-infection between a patient and subsequent patients.

The hollow tube 2 may be substantially cylindrical (which includes cylindrical), but this is not essential and the hollow tube 2 may have any cross-section which is large enough to encompass the first and second sets of valves 3, 5 whilst permitting passage of a substantially cylindrical object 4 of a desired size. For example, the hollow tube 2 may have a cross-section which is elliptical, square, rectangular, pentagonal, hexagonal, or any other regular or irregular shape. The hollow tube 2 may be made of any material which prevents passage of gas and/or fluid, and should also be made from a material which may be effectively sterilised for packaging. Although the valve assembly 1 is likely to be single use in practice, it must be sterilised at least once after manufacture before it is packaged. For example, the hollow tube 1 may be formed from plastic materials such as, for example, polycarbonate, high density polyethylene or any other suitable medical grade plastic or polymer.

The first and second sets of valves 3, 5 preferably form a substantially airtight (alternatively fluid-tight) seal around the substantially cylindrical object 4. The first set of valves 3 may take the form of a single valve, but more generally may include two or more valves. Similarly, the second set of valves 5 may take the form of a single valve, but more generally may include two or more valves. For gastroscopy and/or colonoscopy, the first set of valves 3 may preferably include two valves and the second set of valves 5 may preferably include 2 valves. However, in some applications and use cases, more or fewer valves may be necessary to achieve a complete seal against an infectious agent. The number of valves in the first and second sets of valves 3, 5 may be equal, for example 2 by 2, 3 by 3, 4 by 4 and so forth. Alternatively, the number of valves in the first and second sets of valves 3, 5 may be unequal, for example 2 by 3, 3 by 2, 2 by 4 and so forth.

The chamber, or “air lock” or “biocidal zone” located between the two sets 3, 5 of valves may neutralise infectious agents using other methods in addition to, or instead of, UV light 11. For example, the chamber 6 may be connected to a source of suction (negative pressure) in order to remove fluids and/or aerosolised particles. A filter may be connected between the chamber 6 and the source of suction. In other examples, the chamber may also include a port through which gas and/or liquid may be introduced into the chamber 6, and the introduced gas and/or liquid then exits the chamber via a further port taking fluids and/or aerosolised particles from the patient with it. In another example, the chamber 6 could be constantly washed with a rinse solution which is pumped through the chamber 6, for example soapy water or another biocidal solution.

The size (diameter) of the substantially cylindrical object 4 which may be accommodated will depend on the intended application of the first valve assembly 1. For example, the size may be similar for upper and lower gastrointestinal (GI) endoscopies, but may need to be larger for endotracheal intubation.

### Second Valve Assembly

Referring also to FIG. 2, a schematic cross-section of a second valve assembly 12 is shown.

The second valve assembly 12 is the same as the first valve assembly 1, except that the wall of the hollow tube 2 within the chamber includes a first port 13 which connects the chamber 6 to the exterior of the hollow tube 2. The first port 13 is configured for connection to a fluid flow path 14 for supply and/or extraction of a fluid (gas and/or liquid). For example, the first port 13 may receive a fluid flow path 14 in the form of a tube or pipe formed from suitable material such as, for example, polyvinyl chloride (PVC), reinforced PVC, or similar materials. In some examples, the first port 13 may include, or take the form of, an extension and/or extrusion 15 of the hollow tube 2 coinciding with the chamber 6, whilst in other examples the first port 13 may simply be a break or hole in the wall of the portion of the hollow tube 2 coinciding with the chamber 6

With the exception of the first port 13, the chamber 6 defined between the first and second sets of valves 3, 5 remains substantially airtight (fluid-tight) when a cylindrical object 4 is received through the hollow tube 2.

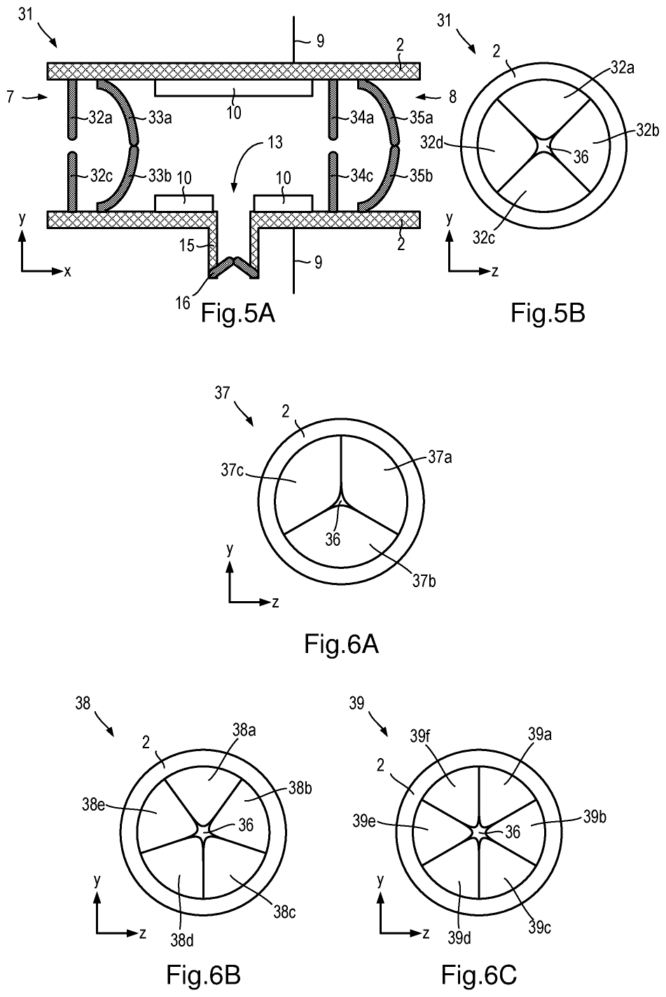

The first port 13 is preferably a first valved port. For example, the first port 13 may include a valve 16 configured to close the first port 13 when the first port 13 is disconnected and/or when the first port 13 does not receive a fluid flow path 14. The valve 16 may take the form of, for example, a duck billed valve (FIGS. 5a, 9A to 10B), one or more lumens, or any other type of valve suitable to provide the function of closing the first port 13 in the absence of connection to a fluid flow path 14. In some examples, the first port 13 may include a valve 16 which is a one-way valve configured to permit fluids (gas/liquid) to exit the chamber 6 via the first port 13 whilst preventing any backflow from the fluid flow path 14 into the chamber 6.

In use, one end of the fluid flow path 14, for example a pipe or tube, is connected to the first port 13, whilst the other end of the fluid flow path 14 is connected to a source of suction 17. The source of suction 17 may take the form of, for example, a pump, a suction canister (connected in turn to a pump), or similar devices capable of applying a negative pressure to the first port 13. The source of suction 17 may be a local source, for example a dedicated stand-alone pump, or may be a non-local source, for example a wall suction system provided to multiple rooms within a hospital. In either case, a suction canister (not shown) may be provided between the source of suction 17 and the first port 13 or filter 18. A filter 18 should be used when the source of suction 17 is common to multiple areas/rooms and/or vents to atmosphere.

A filter 18 configured to remove aerosolised particles may be placed in the fluid flow path 14 between the first port 13 and the source of suction 17, so that a fluid 19 which enters the source of suction 17 is filtered or substantially filtered of infectious agents. Removing particles may mean entrapping particles, biocidal chemical reagents, or any other mechanism suitable for removing, killing, denaturing or otherwise reducing an infectious agent such as a virus or a bacterium. Examples of filters 18 may include, without limitation, a high-efficiency particulate air (HEPA) filter, or an ultra-low particulate air (ULPA) filter. In general, the filter 18 may be configured to remove particles having a dimension greater than or equal to 500 nm, more preferably greater than or equal to 300 nm, and most preferably to remove particles having a dimension greater than or equal to 125 nm. The filter 18 may be specifically configured to remove a particular type and/or variety of infectious agent, for example, to remove virus molecules in general, to remove COVID-19, and so forth.

Waste fluid 20 in the form of gas and/or liquid pumped out of the chamber 6 by the source of suction 17 may be stored in a containment vessel (not shown), or if the filter 18 is included and is sufficiently effective, waste fluid 20 may simply be vented or exhausted to atmosphere or a drain as appropriate.

Optionally, the second valve assembly 12 may include one or more UV sources 10, although UV sources 10 are not essential for the second valve assembly 12.

### Third Valve Assembly

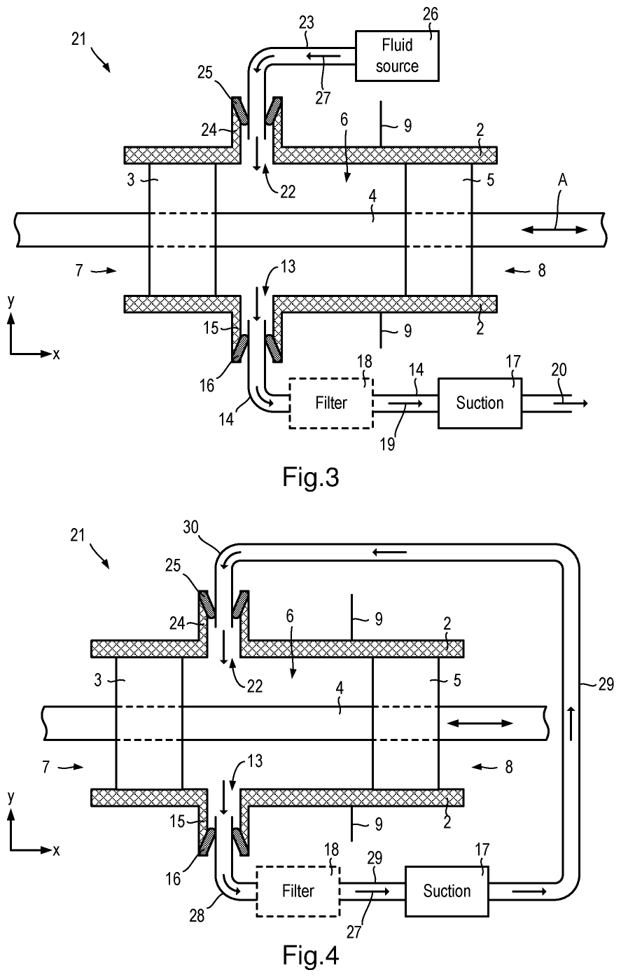

Referring also to FIG. 3, a schematic cross-section of a third valve assembly 21 is shown.

The third valve assembly 21 is substantially the same as the second valve assembly 12, except that the third valve assembly 21 additionally includes a second port 22 connecting the chamber 6 to the exterior of the hollow tube 2. The second port 22 is configured for connection to a second fluid flow path 23. For example, the second port 22 may receive a second fluid flow path 23 in the form of a tube or pipe formed from suitable material such as, for example, polyvinyl chloride (PVC), reinforced PVC or similar materials. In some examples, the second port 22 may include, or take the form of, an extension and/or extrusion 24 of the hollow tube 2 coinciding with the chamber 6, whilst in other examples the first port 13 may simply be a break or hole in the wall of the portion of the hollow tube 2 coinciding with the chamber 6.

With the exception of the first and second ports 13, 22, the chamber 6 defined between the first and second sets of valves 3, 5 remains substantially airtight (fluid-tight) when a cylindrical object 4 is received through the hollow tube 2.

The second port 22 is preferably a second valved port. For example, the second port 22 may include a valve 25 configured to close the second port 22 when the second port 22 is disconnected and/or when the second port 22 does not receive the second fluid flow path 23. The valve 25 may take the form of, for example, a duck billed valve (FIGS. 5a, 9A to 10B), one or more lumens, or any other type of valve suitable to provide the function of closing the second port 22 in the absence of connection to the second fluid flow path 23. In some examples, the second port 22 may include a valve 25 which is a one-way valve configured to permit fluids (gas/liquid) to enter the chamber 6 via the second port 22 whilst preventing any backflow from the chamber 6 into the second fluid flow path 23.

In use, the first port 13 is connected to a source of suction 17 as described in relation to the second valve assembly 12. In some examples, the second port 22 may also be connected to the same, or a different, source of suction 17, in order to provide additional and/or more homogenous removal of fluids and/or aerosolised particles from the chamber 6.

In other examples, and as illustrated in FIG. 3, one end of the second fluid flow path 23 may be connected to the second port 22 whilst the other end of the second fluid flow path 23 is connected to a fluid source 26 which supplies a fluid 27.

The fluid source 26 may supply a fluid 27 in the form of a gas, for example the fluid source 26 may simply be an inlet for atmosphere, or the fluid source may be a pressurised bottle or canister, a pump, a compressor or so forth. Alternatively, the fluid source 26 may supply a liquid from a reservoir, for example water including a biocidal, viricidal and/or anti-bacterial substance or composition such as soap. Depending on the quality of sealing and the possibility of ingestion by a patient, alternative biocidal fluids such as ethyl alcohol, chlorine and chlorine containing compounds, paracetic acid and so forth could be considered for some applications. In this way, the chamber 6 of the third valve assembly 21 may be continuously flushed/rinsed/cleaned to remove and/or destroy infectious agents from the cylindrical object 4, without impending insertion and/or withdrawal of the cylindrical object 4 into/out of a patient.

In the same way as the second valve assembly 12, the third valve assembly 21 may optionally also include one or more UV light sources 10.

Referring also to FIG. 4, an alternative, second configuration of the third valve assembly 21 in use is illustrated.

Instead of connecting the first port 13 to a source of suction 17 and the second port 22 to a fluid source 26, the first port 13 may be connected to a first end 28 of a closed fluid flow path 29 and the second port 22 may be connected to a second end 30 of the closed fluid flow path 29. A source of suction 17, for example a pump, is connected within the closed fluid flow path 29. In effect, an inlet to a pump may provide a source of suction 17 whilst an outlet of the same pump provides a fluid source 26.

A fluid 27 may be filled into the chamber 6 and the closed fluid flow path 29 (for example using additional valves not shown here) and then circulated through the chamber 6. In the case that the fluid 27 is an inert gas or liquid, some form of treatment will be required at a point around the closed fluid flow path 29, for example the filter 18, or an alternative such as a UV treatment unit (not shown), a heat-treatment unit (not shown), a plasma treatment unit (not shown) and so forth. In the case that the fluid 27 is biocidal, for example a liquid having, or including, a biocidal, viricidal and/or anti-bacterial substance or composition, additional filtering or treatment of the re-circulated fluid 27 may be optional.

### Fourth Valve Assembly

Referring also to FIGS. 5A and 5B, a fourth valve assembly 31 is shown. FIG. 5A is a schematic cross-section of the fourth valve assembly 31 along a central axis (parallel to x in the illustrations), and FIG. 5B is a view of the first opening along the central axis.

The fourth valve assembly 31 is an implementation of the second valve assembly 12, which includes UV light sources 10 and which includes two valves in each of the first and second sets of valves 3, 5.

In the fourth valve assembly 31, the first set of valves 3 includes a first septum valve 32 in series with a first duck billed valve 33 in a direction x from the first opening 7 towards the second opening 8. Similarly, the second set of valves includes a second septum valve 34 in series with a second duck billed valve 35 in a direction x from the first opening 7 towards the second opening 8.

The first septum valve 32 is formed from four leaves 32a, 32b, 32c, 32d equally sized and equi-angularly spaced about the centre of the hollow tube 2. Each of the leaves 32a, 32b, 32c, 32d is formed from a resilient material, for example rubber, silicone rubber or comparable materials. Typically a small gap 36 is left at the centre where the leaves 32a, 32b, 32c, 32d do not meet perfectly. The first septum valve 32 may have more or fewer than four leaves, for example three, five, six or more leaves.

Referring also to FIG. 6A, a three-leaved first septum valve 37 is shown having three equally sized and equi-angularly spaced leaves 37a, 37b, 37c.

Referring also to FIG. 6B, a five-leaved first septum valve 38 is shown having five equally sized and equi-angularly spaced leaves 38a, 38b, 38c, 38d, 38e.

Referring also to FIG. 6C, a six-leaved first septum valve 39 is shown having six equally sized and equi-angularly spaced leaves 39a, 39b, 39c, 39d, 39e, 39f.

In general, the more leaves the first septum valve 32, 37, 38, 39 has, the better the seal that may be obtained about the cylindrical object 4, at the cost of greater complexity and fragility.

The second septum valve 34 is preferably configured identically to the first septum valve 32, and may have the same number of leaves. However, there is no practical reason why the first and second septum valves 32 could not have differing numbers of leaves.

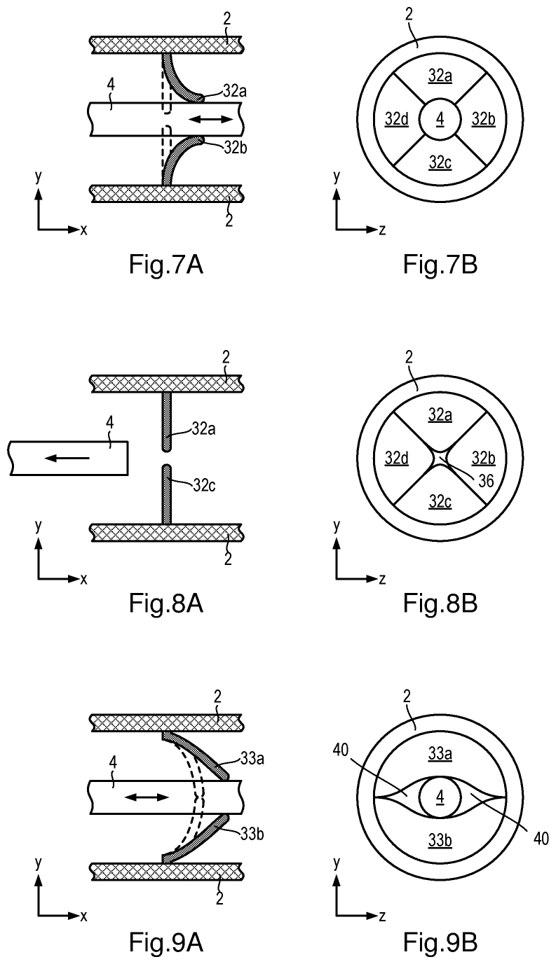

Referring also to FIGS. 7A to 10B, the cooperation of the pairs of septum valves 32, 34 and duck billed valves 33, 35 to maintain a seal containing fluids or aerosolised particles from the patient side of a barrier 9 shall be explained.

Referring in particular to FIGS. 7A and 7B, when a cylindrical object 4 is passed through the first septum valve 32, the resilient leaves 32a, 32b, 32c, 32d deform to form a seal around the circumference of the cylindrical object 4. The resilient leaves 32a, 32b, 32c, 32d will form a seal whether the cylindrical object is moving towards the patient side of the barrier 9 (positive x direction as shown) or towards the clinician side of the barrier 9 (negative x direction as shown). Depending on the friction between the substantially cylindrical object 4 and the leaves 32a, 32b, 32c, 32d, the leaves 32a, 32b, 32c, 32d may temporarily move apart from one another and/or invert when the direction of travel is reversed, but a seal will be quickly re-established after a short travel of the substantially cylindrical object 4.

Referring in particular to FIGS. 8A and 8B, when the substantially cylindrical object 4 is entirely removed, the gap 36 between the leaves 34a, 34b, 34c, 34d remains, and consequently a single septum valve on its own may not provide sufficient containment of potentially infectious aerosolised particles.

Although described in relation to the first septum valve 32 having four leaves 32a, 32b, 32c, 32d, the explanations hereinbefore are equally applicable to the second septum valve 34, or to the first or second septum valve 32, 35, 37, 38, 39 having any number of leaves.

In order to provide sealing when the substantially cylindrical object 4 is removed, the first set of valves 3 includes a first duck billed valve 33 and the second set of valves 5 includes a second duck billed valve 35. A duck billed valve 33, 35 includes two sections 33a, 33b, 35a, 35b which look generally like the two halves of a duckbill, but which are generally more rounded as illustrated in the figures. The two sections 33a, 33b, 35a, 35b of each duck billed valve are formed from a resilient, deformable material such as rubber, silicone rubber or comparable materials.

Referring in particular to FIGS. 9A and 9B, when a substantially cylindrical object 4 is passed through the first duck billed valve 33, the two sections 33a, 33b are deformed and forced apart, creating a gap 40 on either side of the substantially cylindrical object 4. The gaps 40 are acceptable because the first septum valve 32 will form a seal when the substantially cylindrical object 4 is received through the first set of valves 3.

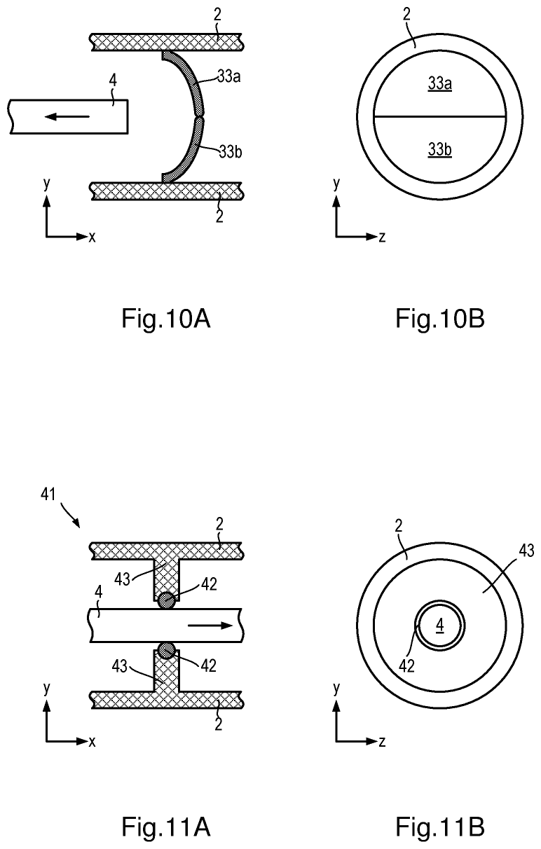

Referring in particular to FIGS. 10A and 10b, when the substantially cylindrical object 4 is completely removed, the two sections 33a, 33b of the first duck billed valve 33 are no longer held apart, and elastically recover their undeformed shapes to close the gaps and seal the interior of the hollow tube 2.

In this way, the first set of valves 3 including a first septum valve 32 and a first duck billed valve 33 may provide a substantially airtight seal both when a substantially cylindrical object 4 is received through the first set of valves 3 (via the first septum valve 32) and when the substantially cylindrical object 4 is removed (via the first duck billed valve 33). Although these functions have been explained in relation to the first set of valves 3, the second set of valves 5 of the fourth valve assembly 31 is configured in the same way as the first set of valves 3, and the functioning of the second set of valves is consequently the same.

Although with perfect valves a single set might be sufficient to allow passage of a substantially cylindrical object 4, for example an endoscope, whilst containing aerosolised particles generated on a patient side of a barrier 9, in practice the second (inner) set of valves 5 will leak a small amount of fluid and/or aerosolised particles. Using the valve assemblies 1, 12, 21, 31 described hereinbefore, any such leakage will be contained in the chamber 6, whilst infectious agents therein are destroyed and/or removed by the UV light source(s) 10 and/or via the first port 13.

Valves of the first and second sets 3, 5 are not limited to septum and duck billed types, and any valves capable of allowing passage of a substantially cylindrical object 4 through the hollow tube 2 may be used.

For example, referring also to FIGS. 11A and 11B, an O-ring valve 41 is shown.

The first set of valves 3 and/or the second set of valves 5 may include one or more O-ring valves 41. The O-ring valve 41 includes an annulus 42 formed of resilient material such as rubber, silicone rubber or comparable materials, and has an inner diameter which is just too small to admit the substantially cylindrical object 4. The substantially cylindrical object 4 may pass through the O-ring valve 41 by slightly compressing the annulus 42, thereby forming a seal about the substantially cylindrical object 4. When the substantially cylindrical object 4 is withdrawn entirely, the hole in the annulus 42 would allow aerosolised particles to pass, and therefore O-ring valves 41 are preferably used in conjunction with at least one other type of valve such as a duck billed valve. Annuli 42 of varying sizes may be used by including a support annulus 43 to fill the space between interior surfaces of the hollow tube 2 and the annulus 42 of the O-ring valve 41.

### Fifth Valve Assembly

The first to fourth valve assemblies 1, 12, 21, 31 use paired sets of valves 3, 5 to define a chamber 6 (or air-lock), and address any leakage of aerosolised particles through the inner (second) set of valve 5 by sterilising, evacuating and/or continuously rinsing the chamber 6.

Another option is to setup an airflow system to remove any aerosolised particles which are able to pass through an inner set of valves of a hollow tube 2. The airflow system may be setup to oppose escape of gas/aerosolised particles originating from the patient side of a barrier 9. At least one mechanical valve is preferred for airflow type valve assemblies, in order to help avoid escape of aerosolised particles from a patient side of a barrier 9 during overpressure events such as, for example, sneezing, coughing, flatulence and so forth.

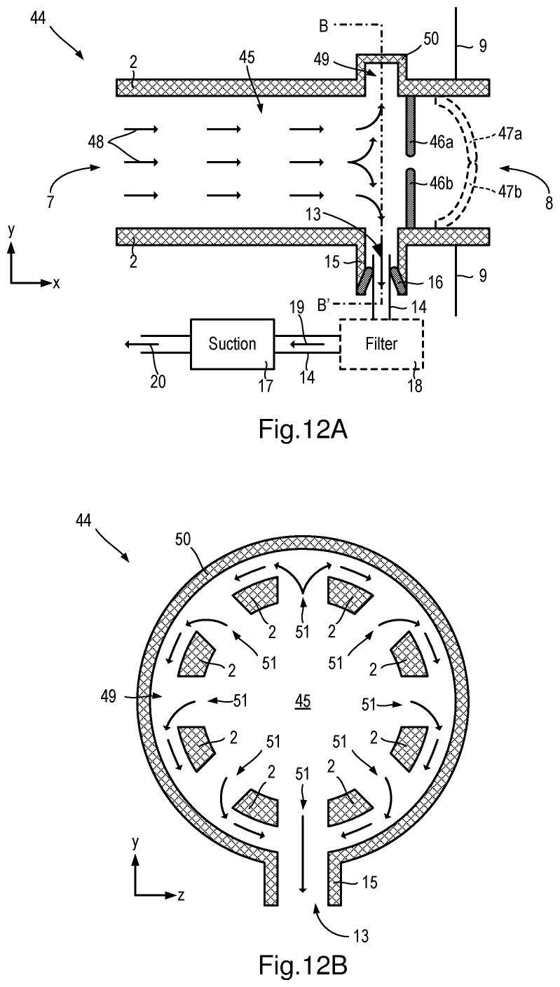

Referring also to FIG. 12A, a schematic cross-section of a fifth valve assembly 44 is shown along a plane containing a central axis of the hollow tube 2.

Referring also to FIG. 12B, a schematic cross-section of a fifth valve assembly 44 is shown along the line labelled B-B′ in FIG. 12A.

The fifth valve assembly 44 includes a hollow tube 2 which is substantially the same as the hollow tube 2 of the first to fourth valve assemblies 1, 12, 21, 31. The hollow tube 2 has a first opening 7 on a clinician side of an airtight barrier 9, and a second opening 8 on a patient side of the barrier 9. The interior of the hollow tube 2 running from the first opening 7 to the second opening 8 defines a channel 45. A third set of one or more valves 46, 47 is arranged within the channel 45 of the hollow tube 2, at or close to the second opening 8. The third set of valves 46, 47 is configured to permit sliding passage of a substantially cylindrical object 4. In the example illustrated in FIG. 12A, the third set of valves 46, 47 includes at least a third septum valve 46 and optionally includes a duck billed valve 47. The third septum valve 46 may be exchanged for an O-ring valve 41, or any other valve suitable for forming a seal around a substantially cylindrical object 4 as it is passed through the channel 45. Similarly, any valve capable of allowing passage of a substantially cylindrical object 4 whilst forming a seal when the substantially cylindrical object 4 is removed may optionally be used instead of the third duck billed valve 47.

The fifth valve assembly 44 also includes a first port 13 which is configured substantially as described hereinbefore. The first port 13 connects the channel 45 to the exterior of the hollow tube and is configured for connection to a first fluid flow path 14 for extraction of gasses (and aerosolised particles suspended therein) from the channel 45. The first port 13 is disposed within the channel 45 at a position between the third set of valves 46, 47 and the first opening 7.

In some examples, the first port 13 may communicate directly with the channel 45, for example as described in relation to the second, third and/or fourth valve assemblies 12, 21, 31. However, symmetry of the airflow 48 through the channel 45 may be improved by having the first port 13 communicate instead with an outer annular cavity 49 defined between an outer annulus 50 and the hollow tube 2. The outer annular cavity 49 communicates with the channel 45 via a number of fenestrations 51 spaced around the circumference of the hollow tube 2.

In use, one end of a fluid flow path 14, for example a pipe or tube, is connected to the first port 13, as described hereinbefore in relation to the second or third valve assemblies 12, 21. However, for use with the fifth valve assembly 44, the negative pressure provided by the source of suction 17 should be sufficient that any aerosolised particles which leak through the third set of valves 46, 47 are unable to reach the first opening 7 due to the rate of flow of air in through the first opening 7, along the channel and out via the fenestrations 51, outer annular cavity 49 and first port 13. This may also be referred to as an “airflow” valve.

Optionally, the fifth valve assembly 44 may include one or more UV sources 10 mounted to the interior surfaces of the hollow tube 2 and within the channel 45, although UV sources 10 are not essential for the fifth valve assembly 44.

The third set of valves 46, 47 may include a single valve, for example the third septum valve 46, or the third set of valves 46, 47 may include two, three, four or more valves, each configured to permit the sliding passage of a substantially cylindrical object 4 through the fifth valve assembly 44. The third septum valve 46, and/or other septum valves included in the third set of valves 46, 47, may each include three, four, five or six leaves, or more than six leaves.

In order to provide sufficient airflow to avoid escape of aerosolised particles, a source of suction 17 connected to the first port 13, for example a pump, may typically need to provide a pressure differential of between about 10 and 500 mmHg (1.3 and 67 kPa), or higher). For many medical applications, the necessary pressure range for the source of suction may be between 20 and 100 mmHg (2.7 and 13.3 kPa).

### Sixth Valve Assembly

An alternative way to view to the fifth valve assembly 44 is that it may be the same as the second valve assembly 12, except with the first set of valves 3 omitted and the placement of the first port 13 controlled to be next to the second set of valves 5.

In a similar way, the third valve assembly 21 could be modified to omit the first set of valves 3, to locate the first port 13 close to the second set of valves 5 and to locate the second port 22 close to the first opening 7. Air could be pumped in through the second port 22 and sucked out through the first port 13 to enhance the air-flow along the channel 45 towards the first port 13. Such a modification may utilise features to shape the airflow 48 and provide improved uniformity compared to a simple modification of the third valve assembly 21.

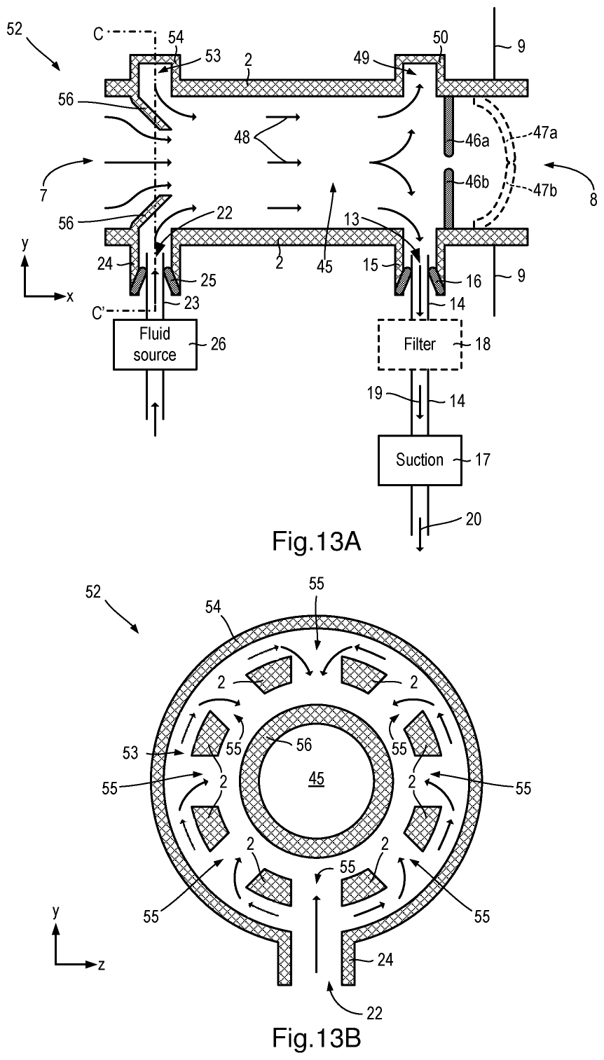

Referring also to FIG. 13A, a schematic cross-section of a sixth valve assembly 52 is shown along a plane containing a central axis of the hollow tube 2.

Referring also to FIG. 13B, a schematic cross-section of the sixth valve assembly 52 is shown along the line labelled C-C′ in FIG. 13A.

The sixth valve assembly 52 is the same as the fifth valve assembly 44, except that the sixth valve assembly 52 also includes a second port 22 connecting the channel 45 to the exterior of the hollow tube 2 and configured for connection to a second fluid flow path 23 for supply of gas into the channel 45. The second port 22 is disposed between the first end 7 and the first port 13, preferably as close as is practical to the first end 7.

In some examples, the second port 22 may communicate directly with the channel 45, for example as described in relation to the third valve assembly 21. However, symmetry of the airflow 48 through the channel 45 may be improved by having the second port 22 communicate instead with a second outer annular cavity 53 defined between a second outer annulus 54 and the hollow tube 2. The second outer annular cavity 53 communicates with the channel 45 via a number of second fenestrations 55 spaced around the circumference of the hollow tube 2.

Gas entering the channel 45 via the second fenestrations would naturally tend to spread in both directions—i.e. towards both first and second ends 7, 8. In some examples this may be acceptable. However, it is preferred that one or more projections 56 should protrude from the inside surfaces of the hollow tube 2 to direct the gas injected via the second fenestrations 55 substantially along the length of the hollow tube 2 towards the first port 13. In the example shown in FIGS. 13A and 13B, there is a single circumferential projection 56 having a truncated conical shape tapering in the direction of the first port 13.

In use, a second fluid flow path 23 is connected to the second port 22 as described hereinbefore, and a fluid source 26 for supplying gas, for example a pump, a compressed canister and so forth. The gas may simply be air drawn in through an inlet. The first port 13 may simply be connected to the filter 18 and gas may be forced through the channel 45 using pressure supplied by the fluid source 26. Preferably, the first port 13 is connected to a source of suction 17 via the filter 18, so that airflow through the channel 45 may be further increased to reduce the possibility of aerosolised particles which pass the third set of valves 46, 47 from escaping through the first opening 7.

### Anti-Aerosolisation Mask

An anti aerosolisation device for gastroscopy, or other upper GI or airway endoscopic procedures, should provide a number of functions. It needs to prevent escape of potentially infectious aerosolised droplets/particles whilst providing access to the mouth for endoscope insertion; and the ability to vent exhaled air from the patient. Optional functions may include, without limitation, oxygen delivery to the patient undergoing the procedure; the ability to suction secretions in the patients' mouth; and the use of an endoscope working channel for endoscopic interventions. Oxygen delivery may not be essential for all potential applications, but is preferable for gastroscopy to ensure that patients receive sufficient oxygen during a procedure.

Referring also to FIGS. 14A to 14C, a mask 57 for covering the mouth and nose of a patient and containing fluids and/or aerosolised particles/droplets is schematically illustrated. FIG. 14A shows a front view whilst FIGS. 14B and 14C show views from opposite sides.

The mask 57 is formed from a gas impermeable material 58, and is configured with a region 59 for forming an airtight seal around a periphery of the mask 57. The gas impermeable material 58 may be a rubber such as, for example, neoprene rubber or silicone rubber, or any other material known for making devices such as masks for gaseous anaesthesia. The region 59 forming a seal may be formed of the same material, and may be configured in a manner which is the same or similar to masks for gaseous anaesthesia. The mask 57 includes a valve assembly 60 which may take the form of any one of the first to sixth valve assemblies 1, 12, 21, 31, 44, 52, or variants thereof, described hereinbefore. The hollow tube 2 of the valve assembly 60 passes through the impermeable material 58 of the mask 57 such that the impermeable material 58 corresponds to the barrier 9. A third port 61 connects an inner surface 62 of the mask 57 to an outer surface 63 of the mask 57. A first integrated filter 64 is installed in, or integrated with, the third port 61, and is configured to remove aerosolised particles.

The third port 61 may take the form of a hole in the material 58 of the mask 57, and the first integrated filter 64 may be joined to, or integrated with, the material 58 of the mask 57. The inner surface 62 of the mask 57 is the surface configured to face a patient in use and the outer surface 63 is the surface configured to face away from a patient in use. The first opening 7 of the valve assembly 60 opens to the outer surface 63 whilst the second opening 8 opens to the inner surface 62. The hollow tube 2 of the valve assembly 60 is integrated with, or connected to, the material 58 of the mask 57 so that a join between the mask 57 and the valve assembly 60 is air-tight.

Removing aerosolised particles may mean entrapping particles, and the first integrated filter 64 may take the form of a high-efficiency particulate air (HEPA) filter or an ultra-low particulate air (ULPA) filter. The first integrated filter 64 may be configured to remove aerosolised particles having a largest dimension greater than or equal to 500 nm, greater than or equal to 300 nm, greater than or equal to 200 nm, greater than or equal to 150 nm, or greater than or equal to 125 nm. The precise dimensions of aerosolised particles which the first integrated filter 64 needs to be able to remove will depend on the application and on the infectious agent of greatest concern. For example, the first filter 64 may be configured to remove virus molecules such as COVID-19. Whilst the COVID-19 virus is approximately 125 nm in size, some studies have suggested that HEPA filters rated to remove 500 nm plus particles may be effective in capturing aerosolised particles such as COVID-19 containing droplets. For example, see Andrew Havics, “The Myth that “Viruses” are Too Small to Be Captured by HEPA Filters”, https://www.researchgate.net/publication/340661151, Technical Report, April 2020 DOI: 10.13140/RG.2.2.32614.78401.

A portion or region (not specifically shown) of the mask 57 including or supporting the valve assembly 60 may include, or be formed from, a flexible material (e.g. more flexible than material 58) to enable positioning and/or adjustment of the position of the valve assembly 60 relative to the mouth or nose of a patient. Depending on the properties of the material 58, such a portion or region (not specifically shown) may be formed from a different material, may be formed by a thinned region of the material 58, or may be unnecessary if the material 58 of the mask 57 has sufficient flexibility. The portion or region (not specifically shown) of the mask including or supporting the valve assembly 60 may optionally be formed from, a material which is transparent as well as flexible, in order to allow medical staff to see the mouth and nose of the patient. In some examples, the material 58 of the mask 57 may be transparent.

The third port 61 need not take the form of, for example, a hole in the material 58 which is spanned by the first integrated filter 64. For example, the third port 61 may take the form of a one-way valved port (not shown) which is configured to permit gas to pass in a direction from the inner surface 62 to the outer surface 63. The first integrated filter 64 may be integrated with, or installed within, such a one-way valved port (not shown).

The mask 57 may also include a fourth port 65 connecting the inner surface 62 of the mask 57 to the outer surface 53 of the mask 57 for delivery of oxygen to a patient wearing the mask. The fourth port 65 may take the form of a valved port, and may include for example a duck billed valve 66 configured to close the fourth port 65 when the fourth first port is disconnected and does not receive a tube or pipe for supplying oxygen. Any type of valve capable of closing off the fourth port 65 when it is not in use may be used instead of the duck billed valve 66, for example a valved fourth port 65 may include one or more lumens (not shown) configured to close the fourth port 65 when it is not receiving a tube or pipe for supplying oxygen. In some examples, the fourth port 65 may include, or take the form of, a one-way valved port (not shown) configured to permit gas to pass in a direction from the outer surface 63 to the inner surface 62.

The mask 57 may also include a suction catheter 67 integrated with the mask 57 and configured for suction of fluids through the mask 57. The suction catheter 67 may be integrated with, or connected to, the material 58 of the mask 57 so that a join between the mask 57 and the suction catheter 67 is air-tight. In the same way as for the valve assembly 60, a portion or region (not specifically shown) of the mask 57 including or supporting the suction catheter 67 may include, or be formed from, a flexible material to enable positioning and/or adjustment of the position of the suction catheter relative to the mouth or nose of a patient. In the same way as for the valve assembly 60, the portion or region (not specifically shown) of the mask 57 including or supporting the suction catheter 67 may include, or be formed from, a transparent flexible material. The opening of the suction catheter 67 to the outer surface 63 may be terminated with a valve, for example a duck billed valve 68, to seal the suction catheter 67 when it is not in use and disconnected from a tube or pipe (not shown) providing suction.

The hollow tube 2 of the valve assembly 60 may include (e.g. terminate in) or be connected to a mouth guard 69 which is received into the mouth of a patient in use. The purpose of the mouth guard 69 is to hold the patients' mouth open so as not to impede insertion of an endoscope. For example, the mouth guard 69 may be inserted between the patient's upper and lower teeth. This may assist by holding their mouth open for easier insertion of the scope. The mouth guard 69 may also serve to prevent a patient biting on the scope during the procedure, which could impede the procedure and/or potentially damage the scope and/or teeth of the patient.

The mask includes adjustable straps 70 which in use will be placed around the head of the patient to ensure a secure seal by the seal region 59.

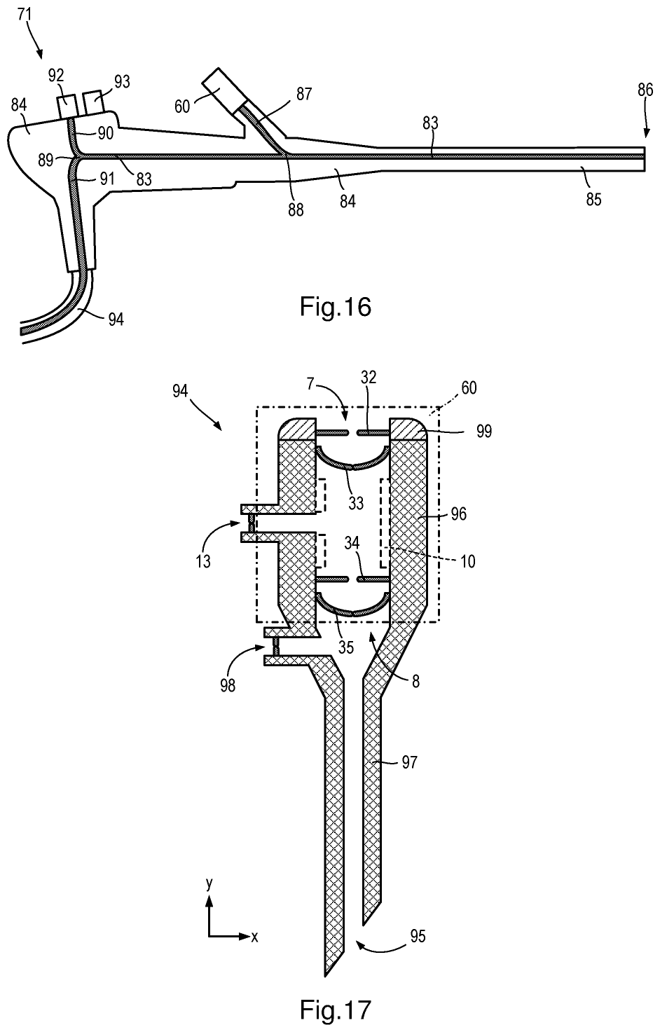

In use the mask 57 is fitted over the mouth and nose of a patient, and an endoscope 71 (FIG. 16) is inserted through the valve assembly 60 and into the mouth or nose of the patient. If the valve assembly 60 includes first and/or second ports 13, 22, then these will be connected as described hereinbefore once the mask 57 has been fitted to the patient. An oxygen supply (not shown) may be connected to the fourth port 65 if required. If required, a source of suction such as a pump may be connected to the suction catheter 67.

In this way, the patient's mouth and nose may be accessed for endoscopy whilst containing any aerosolised particles/droplets. The exhalations of the patient are filtered by the first integrated filter 64 so that gas escaping the mask 57 through the third port 61 does not carry aerosolised particles/droplets with it.

### Anti-Aerosolisation Garment

An anti aerosolisation device for colonoscopy, or other lower GI endoscopic procedures, should provide a number of functions. It needs to prevent escape of potentially infectious aerosolised particles/droplets whilst providing access to the anus for endoscope insertion and preferably also for digital rectal examination.

Referring also to FIGS. 15A and 15B, a garment 72 for covering the anus and pelvic region of a patient and containing fluids and/or aerosolised particles/droplets is schematically illustrated. FIG. 15A shows a rear view whilst FIG. 15B show a schematic cross-section along the line labelled D-D′ in FIG. 15A.

The garment 72 formed from a gas impermeable material 73 such as, for example, neoprene rubber or silicone rubber, any other material suitable for forming the mask 57 described hereinbefore and/or any flexible and impervious (to gas/fluid flow) medical grade plastic. The garment 72 is configured for forming a first airtight seal 74 around the waist of a patient and second and third airtight seals 75, 76 around the left and right legs of a patient, so as to enclose a volume between the patient, the garment 72 and the first to third airtight seals 74, 75, 76. Garments 72 may be produced in a range of sizes to fit patients of different sizes.

The garment 72 includes the valve assembly 60, which may take the form of any one of the first to sixth valve assemblies 1, 12, 21, 31, 44, 52 or variants thereof described hereinbefore. The hollow tube 2 of the valve assembly 60 passes through the impermeable material 73 of the garment 72 such that the impermeable material 73 corresponds to the barrier 9. A fifth port 76 connects an inner surface 77 of the garment 72 to an outer surface 78 of the garment 72. A second integrated filter 79 is installed in, or integrated with, the fifth port 76, and is configured to remove aerosolised particles. The second integrated filter 79 may be configured in any way described hereinbefore for the first integrated filter 64.

The fifth port 76 may take the form of a hole in the material 73 of the garment 72, and the second integrated filter 79 may be joined to, or integrated with, the material 73 of the garment 72. The inner surface 77 of the garment 72 is the surface configured to face a patient in use and the outer surface 78 is the surface configured to face away from a patient in use. The first opening 7 of the valve assembly 60 opens to the outer surface 78 whilst the second opening 8 opens to the inner surface 77. The hollow tube 2 of the valve assembly 60 is integrated with, or connected to, the material 73 of the garment 72 so that a join between the garment 72 and the valve assembly 60 is air-tight.

A portion or region 80 of the garment 72 including or supporting the valve assembly 60 may include, or be formed from, a flexible material (e.g. more flexible than material 73) to enable positioning and/or adjustment of the position of the valve assembly 60 relative to the anus of a patient. Depending on the properties of the material 73, such a portion or region 80 may be formed from a different material, may be formed by a thinned region of the material 73, or may be unnecessary if the material 73 of the garment 72 already has sufficient flexibility. The portion or region 80 of the garment 72 including or supporting the valve assembly 60 may optionally be formed from, a material which is transparent as well as flexible, in order to allow medical staff to see the anus of the patient in relation to an endoscope being inserted through the valve assembly 60. In some examples, the material 73 of the garment 72 may be transparent.

The fifth port 76 need not take the form of, for example, a hole in the material 73 which is spanned by the second integrated filter 79. For example, the fifth port 76 may take the form of a one-way valved port (not shown) which is configured to permit gas to pass in a direction from the inner surface 77 to the outer surface 78, for example to vent flatulence occurring during a colonscopy. The second integrated filter 79 may be integrated with, or installed within, such a one-way valved port (not shown).

The garment 72 may additionally include a sealed, flexible projection 81 arranged to be positioned proximate to the patient's anus when the garment 72 is worn. The projection 81 has an opening 82 and is formed to project inwards from the inner surface 77 of the garment 72. The projection 81 is configured to receive a finger via the opening 82 in order to perform a digital rectal examination of the patient when the garment 72 is worn. The flexible projection 81 may be positioned within the portion or region 80.

In use the garment 72 is fitted over the pelvic region of a patient, and an endoscope 71 (FIG. 16) is inserted through the valve assembly 60 and into the anus of the patient. If the valve assembly 60 includes first and/or second ports 13, 22, then these will be connected as described hereinbefore once the garment 72 has been fitted to the patient.

In this way, the patient's anus may be accessed for endoscopy (colonoscopy) whilst containing any aerosolised particles/droplets. Any flatulence expelled by the patient will be filtered by the second integrated filter 79 so that gas escaping the garment 72 through the fifth port 76 does not carry aerosolised particles/droplets with it.

### Endoscope

Referring also to FIG. 16, a partial schematic cross-section of an endoscope 71 is shown.

Each of the valve assemblies 1, 12, 21, 31, 44, 60, mask 57 and garment may be used in combination with an endoscope 71. The endoscope 71 may be inserted through the hollow tube 2 and used as normal. However, in some applications and situations, it may be desirable to use one or more working channels 83 of the endoscope 71 to extract samples (e.g. biopsies) and/or insert specialised tools along the working channel 83. In order to prevent escape of potentially infections fluids and/or aerosolised particles/droplets, one or more valve assemblies 60 may be added to the endoscope 71, each forming an airtight seal to a user end of a working channel 83. For example, a valve assembly 60 may be connected to, or integrated with, a port providing access to the working channel 83, may replace a endoscopic biopsy channel seal and so forth. Additionally or alternatively, the valve assembly 60 may be configured for introducing endoscopic consumable instruments such as biopsy forceps into the working channel 83.

In the example of an endoscope 71 shown in FIG. 16, the endoscope includes a control handle 84 which remains outside of a patient and is used by a medical practitioner to control the endoscope 71, and an insertion tube 85 which extends from the control handle 84 to a tip 86 which is inserted into the patient. Many different channels (not shown) may run along the insertion tube 85 to the tip, including optical channels, suction channels, wash channels, and other working channels 83. However, in FIG. 16 a single working channel 83 is illustrated for visual clarity.

Upon entering the control handle 84, an access channel 87 branches from the working channel 83 at a first junction 88. The access channel 87 lead to a port fitted with a valve assembly 60. The working channel 83 continues beyond the first junction 88 to a second junction 89 where it divides into first and second branches 90, 91. The first branch 90 leads to a port 92 for connection of, for example, a suction source, or a source of rinsing solution, and so forth. The endoscope 71 control handle 84 may support additional ports 93 connected to other channels (not shown). The second branch 91 is optional, and may lead via a connecting cable 94 to auxiliary equipment (not shown).

In general, any port or seal of an endoscope 71 which may be used for inserting objects into a working channel 83 and/or removing objects (e.g. samples) from the working channel 83 may be replaced with a valve assembly 60 to prevent escape of potentially infectious aerosolised particles/droplets via such ports or seals.

In use, ports 13, 22 of the valve assembly 60 may be connected to external equipment as described hereinbefore.

### Trocar

Keyhole surgeries are performed endoscopically by insertion of instruments through the ports of trocars inserted into, for example, a patient's abdomen and/or chest cavity. Keyhole surgeries are preferred to open surgeries for many procedures, because trauma to the patient is minimised and patients may recover from the surgery more rapidly. Keyhole surgeries may also be performed more rapidly than open surgeries, allowing increased throughput for elective procedures. However, keyhole surgery, for example laparoscopic surgery, is considered a high risk for aerosolisation, meaning that the potential presence of infectious agents such as COVID-19 has led to an increase in the number of open surgeries because these are considered less of a risk for aerosolisation. This may be detrimental to patients because of the increased trauma, and detrimental to the healthcare provider overall because of the prolonged patient recovery times and decreased throughput.

Valve assemblies 1, 12, 21, 31, 44, 52, 60 according to the present specification may be installed to replace the existing ports on trocars used for keyhole surgery, in order to prevent aerosolised droplets/particles from escaping the trocars (e.g. containing active infectious agents).

Referring also to FIG. 17, a schematic cross-section of a trocar 94 is shown.

Similar to a conventional trocar, the trocar 94 includes a tip 95 connected to a port body 96 by a cannula 97, and includes an insufflation port 98. Unlike a conventional trocar which would be capped by, for example, a single septum valve, the body 96 of the trocar 94 includes a valve assembly 60 and the cannula 97 is accessed via the valve assembly 60. In the example shown in FIG. 17, the valve assembly 60 takes the form of the fourth valve assembly 31, though this could be exchanged for any of the first, second, third, fifth or sixth valve assemblies 1, 12, 21, 44, 52. An uppermost portion 99 of the body 96 which supports the first septum valve 32 may be removable and replaceable so that different size first septum valves 32 may be used, depending on the diameter of an instrument (object 4) which the trocar 94 is intended to be used with. The uppermost portion 99 may be attached to the rest of the body 96 using any suitable removable means such as, for example, screw threading, clamps, clips, cooperating protrusions and apertures, or a combination of such means.

In use, ports 13, 22 of the valve assembly 60 may be connected to external equipment as described hereinbefore.

### Use Methods of the Valve Assembly

Prior to commencing a procedure, the first port 13 of a valve assembly 1, 12, 21, 31, 44, 52, 60, for example installed in a mask 57, garment 72, endoscope 71, trocar 94 and so forth, may be connected to a source of suction 17 using a first fluid flow path 14. Preferably the connection is via a filter 18, unless the exhausted fluid 20 will be stored instead of vented. When a valve assembly 21, 52, 60 includes a second port 22, the second port 22 is connected to a fluid source 26.

A valve assembly 1, 12, 21, 31, 44, 52, 60, for example installed in a mask 57, garment 72, endoscope 71, trocar 94, may be used in any medical procedure in which a substantially cylindrical object 4 is used, in order to maintain a barrier 9 between a patient and one or more medical staff.

Examples of suitable medical procedures include, without being an exhaustive list, endoscopy; colonoscopy; any lower gastro-intestinal endoscopy procedures such as, for example, prostoscopy, rigid and flexible sigmoidosopy, colonoscopy and so forth; any upper gastrointestinal endoscopy procedures such as, for example, gastroscopy, nasendoscopy, enteroscopy, endoscopic ultrasound (EUS), endoscopic retrograde cholangio-pancreatography (ERCP) and so forth; any endoscopic respiratory procedures such as, for example, bronchoscopy, endobronchial ultrasound (EBUS), pharyngoscopy and so forth; keyhole surgery such as, for example, laparoscopy, thoracoscopy, or similar minimally invasive procedures which utilise trocars/ports for access to the body; transrectal ultrasound (TRUS) guided Prostate biopsy (TRUS) procedures; extubation and other manoeuvres or procedures with endotracheal tubes such as, for example, changing ventilators and so forth.

### Modifications

It will be appreciated that many modifications may be made to the embodiments hereinbefore described. Such modifications may involve equivalent and other features which are already known in the design and use of endoscopes, endoscopy equipment, endoscopic ports, trocars and/or any equipment and/or accessories for endoscopy and/or keyhole surgery, and which may be used instead of, or in addition to, features already described herein. Features of one embodiment may be replaced or supplemented by features of another embodiment.

Examples have been described which include UV light sources 10 to kill/denature potentially infectious agents. However, the valve assemblies 1, 12, 21, 31, 44 52, 60 are not limited to use of UV light sources 10, and in alternative implementations one or more heat sources (not shown) may be used to heat the interior of a chamber 6 or a channel 45 to a sufficiently high temperature to kill, destroy or denature potentially infectious agents such as viruses and bacteria. Heat sources (not shown) may be used to heat the interior of a chamber 6 or channel 45 to at least 92 degrees Celsius.

Examples have been described in which a fluid such as a flushing gas and/or a biocidal liquid could be flowed between a first port 13 and a second port 22 of, for example, the third valve assembly 21. In some examples, a suitable fluid may take the form of steam which may be used to sterilise the interior of a chamber 6 or a channel 45.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicant hereby gives notice that new claims may be formulated to such features and/or combinations of such features during the prosecution of the present application or of any further application derived therefrom.

1. A valve assembly comprising:
a hollow tube;
a first set of one or more valves arranged within the hollow tube, the first set of valves configured to permit sliding passage of a substantially cylindrical object;
a second set of valves arranged within the hollow tube and spaced apart from the first set of valves to define a chamber, the second set of valves configured to permit sliding passage of the cylindrical object;
the valve assembly further comprising:
one or more ultraviolet light sources within the chamber; and/or
a first port connecting the chamber to the exterior of the hollow tube and configured for connection to a fluid flow path for supply and/or extraction of fluid.

2. A valve assembly according to claim 1, wherein the valve assembly comprises the first port, the valve assembly further comprising a second port connecting the chamber to the exterior of the hollow tube and configured for connection to a fluid flow path for supply and/or extraction of fluid.

3. A valve assembly according to claim 1, wherein the first set of valves comprises two or more valves and/or the second set of valves comprises two or more valves.

4. (canceled)

5. A valve assembly according to claim 1, wherein the first set of valves comprises at least one septum valve and/or the second set of valves comprises at least one septum valve.

6. A valve assembly according to claim 1, wherein the first set of valves comprises at least one duck-billed valve and/or the second set of valves comprises at least one duck-billed valve.

7. A valve assembly according to claim 1, wherein the first set of valves comprises at least one O-ring and/or the second set of valves comprises at least one O-ring.

8. A valve assembly comprising:
a hollow tube defining a channel between a first opening and a second opening;
a third set of one or more valves arranged within the hollow tube, the third set of valves configured to permit sliding passage of a substantially cylindrical object;
a first port connecting the channel to the exterior of the hollow tube and configured for connection to a fluid flow path for extraction of gas; wherein the first port is disposed between the third set of valves and the first opening.

9. A valve assembly according to claim 8, further comprising a second port connecting the channel to the exterior of the hollow tube and configured for connection to a fluid flow path for supply of fluid, wherein the second port is disposed between the first end and the first port.

10. A valve assembly according to claim 8, wherein the third set of valves comprises two or more valves.

11. A valve assembly according to claim 8, wherein the third set of valves comprises one or more of a septum valve, a cuck-billed valve and an O-ring.

12. (canceled)

13. (canceled)

14. A mask for covering the mouth and nose of a patient, the mask formed from a gas impermeable material and configured to form an airtight seal around a periphery of the mask, the mask comprising:
a valve assembly according to claim 1, wherein the hollow tube of the valve assembly passes through the mask;
a third port connecting an inner surface of the mask to an outer surface of the mask and comprising a first filter, the first filter configured to remove aerosolised particles.

15. A mask according to claim 14, wherein the third port comprises a third one-way valved port configured to permit gas to pass in a direction from the inner surface to the outer surface.

16. A mask according to claim 14, further comprising a fourth port connecting the inner surface of the mask to the outer surface of the mask for delivery of oxygen to a patient wearing the mask.

17. A mask according to claim 14, further comprising a suction catheter integrated with the mask and configured for suction of fluids through the mask.

18. A mask according to claim 14, wherein the hollow tube of the valve assembly comprises a mouth guard or the hollow tube of the valve assembly is connected to a mouth guard.

19. A garment configured for forming a first airtight seal around the waist of a patient and second and third airtight seals around corresponding legs of a patient so as to enclose a volume between the patient, the garment and the first to third airtight seals, the garment formed from a gas impermeable material and comprising:
a valve assembly according to claim 1, wherein the hollow tube of the valve passes through the garment;
a fifth port connecting an inner surface of the garment to an outer surface of the garment and comprising a second filter, the second filter configured to remove aerosolised particles.

20. A garment according to claim 19, further comprising a sealed, flexible projection arranged to be positioned proximate to the patient's anus when the garment is worn, the projection configured to receive a finger in order to perform a digital rectal examination of the patient.

21. An endoscope comprising:
a working channel; and
a valve assembly according to claim 1, wherein the hollow tube forms an airtight seal to a user end of the working channel.

22. A trocar comprising a valve assembly according to claim 1, wherein a cannula of the trocar is configured to be accessed via the valve assembly.

23. (canceled)

24. A method comprising connecting a first port of a valve assembly according to claim 1 to a source of suction.

25. (canceled)
